(19) United States
(12) Patent Application Publication
(10) Pub. No.: US 2009/0270630 A1
(43) Pub. Date: Oct. 29, 2009

(54) PROCESS FOR THE SYNTHESIS OF BENZYLIDENE ROSIGLITAZONE BASE

(76) Inventors: Laszlo Czibula, Budapest (HU); Ferenc Sebok, Mezokovacshaza (HU); Laszlo Dobay, Budapest (HU); Eva Werkne Papp, Budapest (HU); Ida Deutschne Juhasz, Budapest (HU); Judit Nagyne Bagdy, Budapest (HU); Tamasne Uberhardt, Budapest (HU)

Correspondence Address: HAHN & VOIGHT PLLC, 1012 14TH STREET, NW, SUITE 620, WASHINGTON, DC, 20005, US

(21) Appl. No.: 12/302,127

(22) PCT Filed: Jun. 21, 2007
(86) PCT No.: PCT/HU07/00056

§ 371 Date: Nov. 24, 2008

(30) Foreign Application Priority Data
Jun. 23, 2006 (HU) P0600517

(51) Int. Cl. C07D 417/12

(52) U.S. Cl. 546/269.7

(57) ABSTRACT

Process for the synthesis of 5-{4-[N-methyl-N-(2-pyridyl)-amino-ethoxy]-benzylidene}-thiazolidine-2,4-dione (INN name: benzylidene-rosiglitazone) of formula (I), which consist of the following steps:

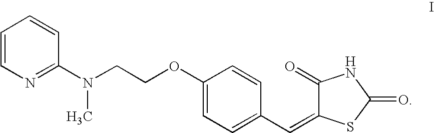

Step a) reaction of 2-chloro-pyridine and 2-(N-methylamino)-ethanol
Step b) reaction of the obtained compound of formula (III) with 4-fluorobenzaldehyde
Step c) reaction of the obtained compound of formula (IV) with thiazolidine-2,4-dione

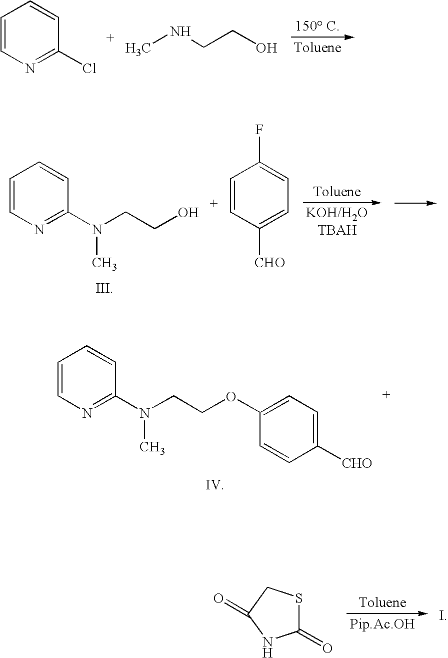

characterized by
dissolving 4-{2-[N-methyl-N-(2-pyridyl)amino]-ethanol of formula (III) obtained in Step a) in toluene and using it in Step b) without isolation;
reacting the solution of compound of formula (III) in toluene in Step b) with 4-fluorobenzaldehyde in the presence of aqueous alkali hydroxide solution and phase transfer catalyst at 25-50° C.;
reacting the solution of the benzaldehyde derivative of formula (IV) obtained in Step b) in toluene in Step c) and isolating the desired product.

The invention relates to a new process for the synthesis of 5-{4-[N-methyl-N-(2-pyridyl)-amino-ethoxy]-benzylidene}-thiazolidine-2,4-dione (INN name: benzylidene-rosiglitazone) of formula (I):

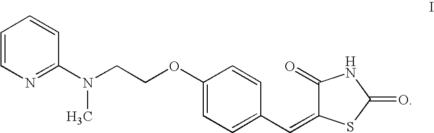

Compound of formula (I)—benzylidene-rosiglitazone—is the key-intermediate of the synthesis of 5-{4-[N-methyl-N-(2-pyridyl)-amino-ethoxy]}-thiazolidine-2,4-dione—INN name: rosiglitazone—, which is the active ingredient of the drug for the treatment of non-insulin dependant diabetes.

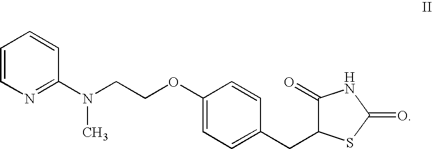

Rosiglitazone of formula (II) was first described in EP 306228 of Beecham. Benzylidene rosiglitazone of formula (I) and the synthesis thereof was also described in this Patent. The first step of the synthesis is the reaction of 2-chloro-pyridine with 2-(methylamino)-ethanol at 150° C. Then the obtained 4-{2-[N-methyl-N-(2-pyridyl)amino]-ethanol} of formula (III) is reacted with 4-fluorobenzaldehyde and NaH in dimethyl formamide (DMF) to furnish {2-[N-methyl-N-(2-pyridyl)amino]-ethoxy}-benzaldehyde of formula (IV). The latter is reacted with thiazolidine-2,4-dione in the presence of piperidinium acetate in toluene to yield the intermediate benzylidene rosiglitazone of formula (I).

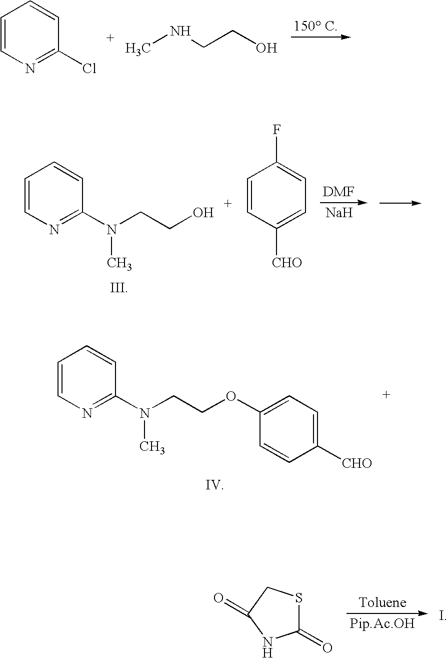

EP 306228 of Beecham does not describe the synthesis of compound of formula (IV), the formation of the ether bond is presented only by analogous example. According to the analogous example the NaH reagent is reacted in DMF at high temperature (80° C.) and the reaction time is 16 h. During the work-up procedure diethyl ether is used, which is extremely explosive and flammable and the obtained oily crude product is purified by chromatography.

WO 01 44240 A1 of Richter describes the synthesis of the benzylidene rosiglitazone intermediate of formula (I) also. The compound of formula (III), which is isolated after work-up with dichloromethane, is reacted with 4-fluorobenzaldehyde in the presence of potassium tert-butoxide—instead of NaH—in DMF under anhydrous conditions, but the applied technique is hardly accomplishable in laboratory scale. The compound of formula (III) is dissolved in DMF together with potassium tert-butoxide (KTB) and the obtained solution is added dropwise in an inert atmosphere to the solution of 4-fluorobenzaldehyde in DMF at 75° C.

It is known, that the solubility of KTB in DMF is poor, therefore the ether bond formation reaction should be carried out in diluted solution at high temperature. The solubility of the product is also poor in DMF, therefore the reaction mixture contains gummy separation and during the work-up it is difficult to handle. Further disadvantages of the application of DMF are: high boiling point, it is difficult to distill and at elevated temperature it is prone to decompose.

Further disadvantage of the process is that the product is isolated after gentle distillation of DMF, the industrial realization of which requires special equipment and the formed intermediate of formula (IV) can be used in the next step only after isolation and purification.

According to WO 02 51823 A1 the compound of formula (III) is reacted with 4-fluorobenzaldehyde in the presence of alkali hydride or hydroxide in a polar aprotic solvent (e.g. DMF, DMSO). The formed aldehyde of formula (IV) is isolated and then reacted with thiazolidine-2,4-dione.

The DMF solvent used for the synthesis of aldehyde of formula (IV) has harmful physiological effect and DMSO raises safety problems. The work-up of the reaction mixture is technologically difficult.

The aim of the invention was to elaborate an expedient process, which fulfils safety provisions, environmentally acceptable and the good quality product can be obtained in high yield.

The basis of our invention is that the disadvantages of the previous procedures can be eliminated if the compound of formula (III) is not isolated, it is extracted with toluene and the toluene solution is reacted in an inert atmosphere with 4-fluorobenzaldehyde in aqueous basic medium in the presence of phase transfer catalyst.

This is surprising, because it was not expected that the reaction would proceed in aqueous basic medium.

According to the above mentioned facts the invention relates to a process for the synthesis of 5-{4-[N-methyl-N-(2-pyridyl)-amino-ethoxy]-benzylidene}-thiazolidine-2,4-dione (INN name: benzylidene-rosiglitazone) of formula (I), which consist of the following steps:
Step a) reaction of 2-chloro-pyridine and 2-(N-methylamino)-ethanol
Step b) reaction of the obtained compound of formula (III) with 4-fluorobenzaldehyde
Step c) reaction of the obtained compound of formula (IV) with thiazolidine-2,4-dione

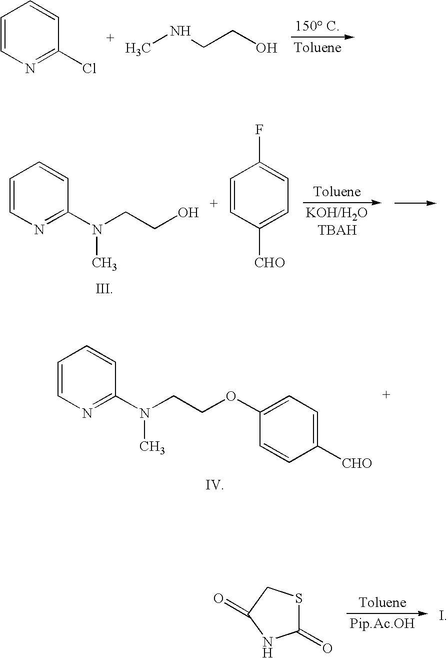

by dissolving 4-{2-[N-methyl-N-(2-pyridyl)amino]-ethanol of formula (III) obtained in Step a) in toluene and using it in Step b) without isolation;
reacting the solution of compound of formula (III) in toluene in Step b) with 4-fluorobenzaldehyde in the presence of aqueous alkali hydroxide solution and phase transfer catalyst at 20-25° C.;
reacting the solution of the benzaldehyde derivative of formula (IV) obtained in Step b) in toluene in Step c) and isolating the desired product.

This process provides the aldehyde of formula (IV) in a reproducible way, in high yield—above 80%.

Compound of formula (IV) is obtained in toluene solution, which can be used in the next chemical step (condensation) directly—without isolating the product—i.e. for the synthesis of benzylidene rosiglitazone of formula (I).

The advantages of the invention are the following:
Only one organic solvent—toluene—is used. The use of DMF, which is carcinogen and pollutes the environment—is eliminated.
The intermediates are obtained in toluene solution and used without isolation in the next steps.
The process of our invention does not apply extremely flammable and explosive reagents (NaH, KTB), but uses aqueous medium.
The high purity benzylidene rosiglitazone of formula (I) is obtained in high yield, in a reproducible way with short reaction time: 3-4 h.

The technological advantage of the process for the synthesis of compound of formula (IV) according to our invention comes to the fore during the reduction of the benzylidene rosiglitazone base of formula I. Namely the reduction to form rosiglitazone of formula (II) can be carried out by using substantially less amount of Pd on charcoal catalyst compared to the previously known procedures (see Reference Example). Hydrogenation of compound of formula (I) obtained by our method to form compound of formula (II) can be carried out by using 20-30% Pd/C —calculated on the weight of compound of formula (I)—in very good yield.

EP 306228 Patent of Beecham describes the hydrogenation of compound of formula (I) to form compound of formula (II) only by analogous example and it applies 150% (!) of catalyst calculated on the weight of compound of formula (I).

This shows the surprising additional effect of our process.

The process according to our invention is illustrated by following not limiting examples.

### EXAMPLE 1

### Step a)

### Synthesis of 2-{N-methyl-N-(2-pyridyl)}-amino-ethanol (III)

Under nitrogen 2-chloro-pyridine (67 g, 55.8 ml, 0.59 mol) and 2-methylamino-ethanol (177.1 g, 188.9 ml, 2.36 mol) were placed into a 500 ml flask provided with stirrer, condenser and thermometer and the mixture was stirred at 140-150° C. for 15 h. After completion of the reaction the excess of 2-methylamino-ethanol was distilled off in vacuum below 110° C. The residue was dissolved in toluene (300 ml, 260.4 g) and washed with 20% sodium chloride solution (3×50 ml, 3×56.5 g). The combined aqueous phases were extracted with toluene (3×70 ml, 3×60.8 g). The combined organic phases were washed with 20% sodium chloride solution (50 ml, 56.5 g). The obtained toluene solution was directly used in the ether formation reaction. The solution contained 84 g (93.5%) of compound of formula (III).

### Step b)

### Synthesis of 4-{2-[N-methyl-N-(2-pyridyl)]-amino-ethoxy}-benzaldehyde (IV)

The toluene solution of compound of formula (III) obtained in Step a) was diluted with toluene (400 ml, 347.2 g), then 4-fluorobenzaldehyde (59.3 ml, 68.5 g, 0.55 mol), tetrabutylammonium hydrogensulfate (18.71 g, 0.055 mol) and a solution of potassium hydroxide (93.5 g, 1.65 mol) in water (93 ml) were added. The reaction mixture was vigorously stirred under nitrogen at 39-41° C. for 3 h. When the reaction was completed, the mixture was diluted with water (300 ml) and stirred at 20-25° C. for 30 min. The organic phase was separated and the aqueous phase was extracted with toluene (100 ml, 86.8 g). The combined organic layers were washed with water (5×250 ml, 5×250 g). The obtained toluene solution was directly used in the next condensation reaction. The solution contained 119 g (84%) of compound of formula (IV).

### Step c)

### Synthesis of 5-{4-[N-methyl-N-(2-pyridyl)-amino-ethoxy]-benzylidene-thiazolidine-2,4-dione (1)

Thiazolidine-2,4-dione (64.83 g, 0.55 mol), piperidine (5.1 ml, 4.41 g, 0.052 mol) and acetic acid (2.96 ml, 3.12 g, 0.052 mol) were added to the toluene solution of compound of formula (IV) obtained in Step b), then the reaction mixture was refluxed at 130-140° C. under nitrogen. The precipitation of the product started after 1 h. When the reaction completed the mixture was cooled to 5-10° C., stirred at this temperature for 3 h, then filtered and washed with cold toluene (150 ml, 130.2 g). The filtered product—without drying—was suspended in methanol (400 ml, 316.4 g) and the suspension was refluxed at 70-75° C. for 30 min. Then the suspension was stirred at 20-25° C. for 2 h and at 5-10° C. for further 2 h. The product was filtered off, washed with cold methanol (100 ml, 79.1 g) and dried below 50° C.

The weight of the obtained wet product: 212.5 g, after drying: 142.9 g (87%).

### Mp: 194-196° C.

### EXAMPLE 2

### Step a)

### Synthesis of 2-{N-methyl-N-(2-pyridyl)}-amino-ethanol (III)

Under nitrogen 2-chloro-pyridine (4.9 kg, 4.1 l) and 2-methylamino-ethanol (13.0 kg, 13.9 l) were placed into a 50 l Lampart duplicator and the mixture was stirred at 140-150° C. for 15 h. When the reaction was completed the excess of 2-methylamino-ethanol was distilled off (6-7 l distillate) in vacuum below 110° C. The residue was cooled to 30° C., dissolved in toluene (22 l, 19.1 kg) and washed with 20% sodium chloride solution (4×3.7 l, 4×4.2 kg). The combined aqueous phases were extracted with toluene (2×5.2 l, 2×4.5 kg). The combined organic phases were washed with 20% sodium chloride solution (3.7 l, 4.2 kg). The obtained toluene solution was directly used in the ether formation reaction.

### Step b)

### Synthesis of 4-{2-[N-methyl-N-(2-pyridyl)]-amino-ethoxy}-benzaldehyde (IV)

The toluene solution of compound of formula (III) obtained in Step a) was placed into 250 l Lampart duplicator and diluted with toluene (30 l, 26.1 kg). Then 4-fluorobenzaldehyde (4.6 l, 5.3 kg), tetrabutylammonium hydrogensulfate (1.4 kg) and a solution of potassium hydroxide (6.9 kg) in water (6.9 l) were added to the solution. The reaction mixture was vigorously stirred under nitrogen at 39-41° C. for 3 h. When the reaction was completed the mixture was diluted with water (22 l) and stirred at 20-25° C. for 30 min. The organic phase was separated and the aqueous phase was extracted with toluene (7.4 l, 6.4 kg). The combined organic layers were washed with water (5×18.5 l). The obtained toluene solution was directly used in the condensation reaction.

### Step c)

### Synthesis of 5-{4-[N-methyl-N-(2-pyridyl)-amino-ethoxy]-benzylidene-thiazolidine-2,4-dione (1)

Thiazolidine-2,4-dione (4.77 kg), piperidine (0.37 l, 0.34 kg) and acetic acid (0.22 l, 0.23 kg) were added to the toluene solution of compound of formula (IV) obtained in Step b), then the reaction mixture was refluxed at 130-140° C. under nitrogen. The precipitation of the product started after 1 h. When the reaction was completed the mixture was cooled to 5-10° C., stirred at this temperature for 3 h, then the product was isolated by centrifugation and washed with cold toluene (11 l, 9.6 kg). The filtered product—without drying—was suspended in methanol (29.5 l, 24 kg) and the suspension was refluxed for 30 min. Then the suspension was stirred at 20-25° C. for 2 h and at 5-10° C. for further 2 h. The product was isolated by centrifugation, washed with cold methanol (7.4 l, 5.9 kg) and dried below 50° C.

The weight of the obtained wet product: 12.1 kg, after drying: 10.6 kg (69% yield calculated on the starting 2-chloro-pyridine).

### Mp: 194-196° C.

### REFERENCE EXAMPLE

### Synthesis of 5-{4-[N-methyl-N-(2-pyridyl)-amino-ethoxy]-benzyl}-thiazolidine-2,4-dione (II)

Benzylidene rosiglitazone base of formula (I) (18.84 g, 0.053 mol) was dissolved in a mixture of acetic acid (375 ml, 393.8 ml) and trifluoroacetic acid (4.25 ml, 6.14 g, 0.053 mol) at 45-50° C. in a 2 l autoclave, then a suspension of 10% Pd on charcoal (5 g) in ion exchanged water (25 ml) was added. After bubbling nitrogen through the mixture the reaction mixture was hydrogenated at 69-71° C. under 5-6 atm pressure for 12-15 h. Then the mixture was cooled to 23-25° C., the air of the autoclave was exchanged for nitrogen, the catalyst was filtered off and washed with acetic acid (40 ml, 42 g). The filtrate was concentrated in vacuum below 60° C. The oily residue was dissolved in a mixture of methanol (100 ml, 79.5 g) and ion exchanged water (50 ml, 50 g) and 7.7 N KOH solution (7.25 ml, 9.58 g, 0.053 mol KOH) was added dropwise. After addition of the basic solution the product started to precipitate. Then the suspension was heated to reflux temperature and was stirred at this temperature for 30 min. Then the suspension was cooled to 0-2° C. with a speed of 1° C./min and stirred for further 3 h. The product was filtered off, washed with ion exchanged water of 0-2° C. (60 ml), methanol of (−10)-(−8)° C. (60 ml, 47.7 g) and dried below 50° C. to yield 16.1 g (84.6%) of the title compound. (The weight of the obtained wet product was 22.3 g.)

Mp: 153-155° C. Purity: min. 95% according to HPLC.

1. Process for the synthesis of 5-{4-[N-methyl-N-(2-pyridyl)-amino-ethoxy]-benzylidene}-thiazolidine-2,4-dione (INN name: benzylidene-rosiglitazone) of formula (I), which consist of the following steps: 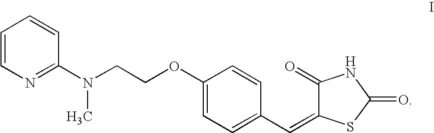
Step a) reaction of 2-chloro-pyridine and 2-(N-methylamino)-ethanol
Step b) reaction of the obtained compound of formula (III) with 4-fluorobenzaldehyde
Step c) reaction of the obtained compound of formula (IV) with thiazolidine-2,4-dione 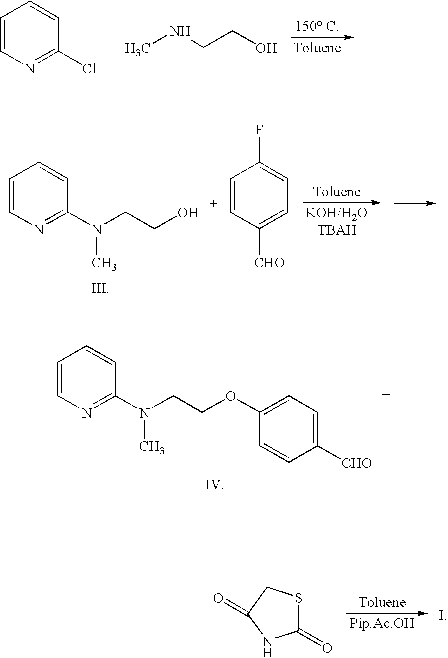
characterized by
dissolving 4-{2-[N-methyl-N-(2-pyridyl)amino]-ethanol of formula (III) obtained in Step a) in toluene and using it in Step b) without isolation;
reacting the solution of compound of formula (III) in toluene in Step b) with 4-fluorobenzaldehyde in the presence of aqueous alkali hydroxide solution and phase transfer catalyst at 25-50° C.;
reacting the solution of the benzaldehyde derivative of formula (IV) obtained in Step b) in toluene in Step c) and isolating the desired product.

2. The process according to claim 1, characterized by using potassium hydroxide as alkali hydroxide in Step b).

3. The process according to claim 1, characterized by using tetrabutylammonium hydrogensulfate (TBAH) as phase transfer catalyst in Step b).

4. The process according to claim 1, characterized by carrying out the reaction in Step b) at 30-45° C.
